(19) United States
(12) Patent Application Publication
(10) Pub. No.: US 2025/0205002 A1
(43) Pub. Date: Jun. 26, 2025

(54) METHOD FOR CONTROLLING A SLAVE DEVICE, CONTROLLED BY A MASTER DEVICE MOVABLE BY AN OPERATOR IN A ROBOTIC SYSTEM FOR MEDICAL OR SURGICAL TELEOPERATION, CLOSE TO MOTION LIMITS OF THE SLAVE DEVICE, AND RELATED ROBOTIC SYSTEM

(71) Applicant: MEDICAL MICROINSTRUMENTS, INC., Wilmington, DE (US)

(72) Inventors: Antonio DI GUARDO, Pisa (IT); Matteo TANZINI, Pisa (IT); Emanuele RUFFALDI, Pisa (IT); Massimiliano SIMI, Pisa (IT)

(21) Appl. No.: 18/851,675

(22) PCT Filed: Mar. 24, 2023
(86) PCT No.: PCT/IB2023/052940

§ 371 Date: Sep. 26, 2024

(30) Foreign Application Priority Data
Mar. 31, 2022 (IT) 102022000006338

(51) Int. Cl. A61B 34/37; A61B 34/00

(52) U.S. Cl. CPC A61B 34/37; A61B 34/77

(57) ABSTRACT

A method for controlling a robotic system slave device for medical or surgical teleoperation, is close to physical motion limits of the slave device. The robotic system includes a master device movable by an operator, controlling a slave device having a surgical instrument. For each master trajectory of the master device, a respective slave target trajectory is determined in a slave reference coordinate system, with slave device movements being reduced by a scale factor with respect to master device movements. Determining the slave trajectory includes defining an edge region and an inner region of a convex volume. When a slave device nominal target trajectory is outside the inner region the modified scale factor is greater than a predetermined maximum scaling factor, the scale factor or the translational offset are dynamically varied so the target slave trajectory remains within the convex volume.

### FIELD OF APPLICATION

The present invention relates to a method and system for controlling a robotic system for medical or surgical teleoperation.

In particular, the invention relates to a method for controlling a slave device, controlled by a master device in a robotic system for medical or surgical teleoperation, close to the movement limits of the slave device.

### DESCRIPTION OF THE PRIOR ART

In the context of master-slave robotic systems for medical or surgical teleoperation, systems with master devices not mechanically constrained to a “master controller” station of the robotic system (i.e., “mechanically ungrounded”, “mechanically unconstrained”), or of the type as shown for example in WO-2019-020407, WO-2019-020408, WO-2019-020409 in the name of the same Applicant, are known.

In a “wheel” master device, without force feedback, and in mono-lateral teleoperation, there is a problem related to what occurs when the master device maps on a corresponding nominal target pose which is not reachable by the slave device, for example because it is outside an allowed workspace for the slave device.

In order to maintain high teleoperation usability, and to maintain intuitive operator behavior, the need arises to provide modified and improved control approaches and algorithms when the slave device is located close to the limits of the allowed workspace and/or when the nominal target pose is outside the aforesaid workspace of the slave device.

A mono-lateral teleoperation is given between a symmetrical N-fold master device and a microsurgical instrument in which there are degrees of freedom of a translational nature (generally 3 directions orthogonal to one another), degrees of freedom of a rotational nature (the space attitude of which can generally be described by 3 successive rotations) and possibly additional degrees describing the state of the microsurgical device, such as the “closure” (or grip).

It is assumed that the symmetrical N-fold master has at least the same number of degrees of freedom as the controlled device. In this context, the mono-lateral teleoperation can be seen as an information flow between master and slave device (as shown for example in FIG. 8).

Since the master device is unconstrained, there is no a priori fixed mapping between the positions of the master device and the slave device. Such a mapping is created in particular moments such as the entry into teleoperation, in which the movement of the slave device “couples” to that of the master device.

In a microsurgical context, with reference to translational degrees of freedom, it is useful that a translational movement of the master device results in a scaled movement of the slave device, so that the user perceives an improvement in positioning accuracy of the master device. The introduction of the scale factor causes the user to perceive better control over the slave device as he/she easily finds a cognitive coherence between the direction and amplitude of the translational trajectories carried out on the master device and the movement of the slave device.

Such a consistency allows the user to close the cognitive control loop given by hand-eye coordination by observing the slave device instead of his/her own hand.

The presence of physical limits, with reference for example to the translational degrees of freedom, requires a particular management of teleoperation close to such limits.

Therefore, in master-slave robotic systems with both constrained and unconstrained master device, the need is felt to define an appropriate teleoperation behavior when the slave device is located close to such limits as well as to optimize the user experience during the change of the teleoperation paradigm.

The known solutions, in the technical field considered, do not allow satisfactorily solving the aforesaid problems and drawbacks.

Therefore, in the technical field considered, there is a strong need to control the enslaved movement of the slave device, depending on the master device, with contrivances and based on control algorithms such as to solve or at least mitigate the aforesaid problems and drawbacks.

### SUMMARY OF THE INVENTION

It is an object of the present invention to provide a method for controlling a slave device, controlled by a master device movable by an operator close to physical motion limits of the slave device, which allows at least partially obviating the drawbacks complained above with reference to the prior art, and responding to the aforementioned needs particularly felt in the technical field considered. Such an object is achieved by a method according to claim 1.

Further embodiments of such a method are defined in claims 2-14.

It is also an object of the present invention to provide a robotic system for medical or surgical teleoperation, configured to be controlled by the aforesaid method. Such an object is achieved by a system according to claim 15.

Further embodiments of such a system are defined by claim 16.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIG. 1 shows a master-slave robotic system for medical or surgical teleoperation, according to an embodiment of the present invention;

FIG. 2 shows in more detail a master device and a slave device, provided in the robotic system in FIG. 1, according to an embodiment of the present invention;

FIG. 3A shows a convex volume definable as a workspace of the slave device;

FIG. 3B shows two regions defined in such a convex volume, an edge region and an inner region, relevant for the purpose of describing the method according to the invention;

FIG. 4 shows a target trajectory S(t) of the slave device, with respect to a nominal target trajectory MT(t) corresponding to the mapping of the master device trajectory in the slave device workspace, when an embodiment of the control method according to the present invention is implemented;

FIG. 5 shows a target trajectory S(t) of the slave device, with respect to a nominal target trajectory MT(t) corresponding to the mapping of the master device trajectory in the slave device workspace, when another embodiment of the control method according to the present invention is implemented;

FIGS. 6A and 6B show geometric relationships related to the mapping between the aforesaid nominal target trajectories MT(t) and target trajectory S(t) in accordance with an embodiment of the method according to the invention:

FIG. 7 shows a target trajectory S(t) of the slave device, with respect to a nominal target trajectory MT(t) corresponding to the mapping of the master device trajectory in the slave device workspace, when another further embodiment of the control method according to the present invention is implemented;

FIG. 8 shows an information flow in a diagrammatic and simplified manner in a master-slave robotic system, between master device and slave device;

FIGS. 9 and 10 show exemplary scale factor reduction functions U (Pp) in respective implementation options of the method according to the present invention.

### DETAILED DESCRIPTION

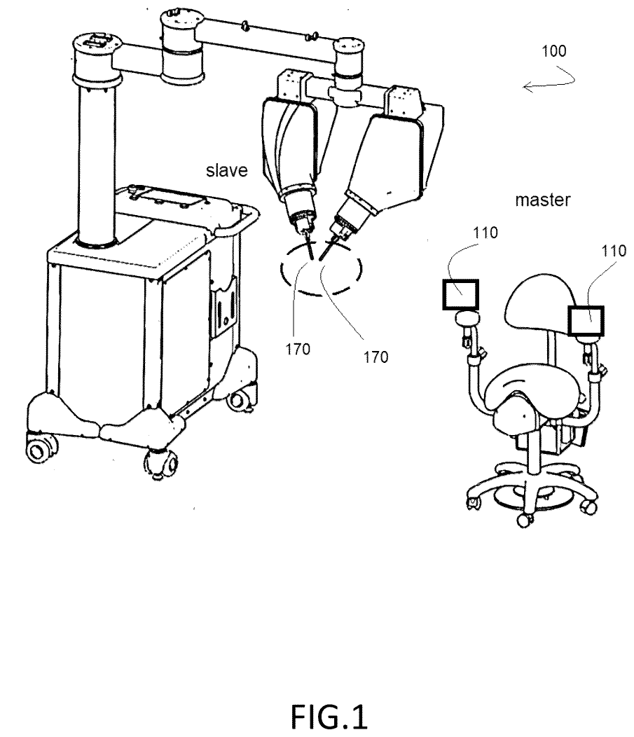

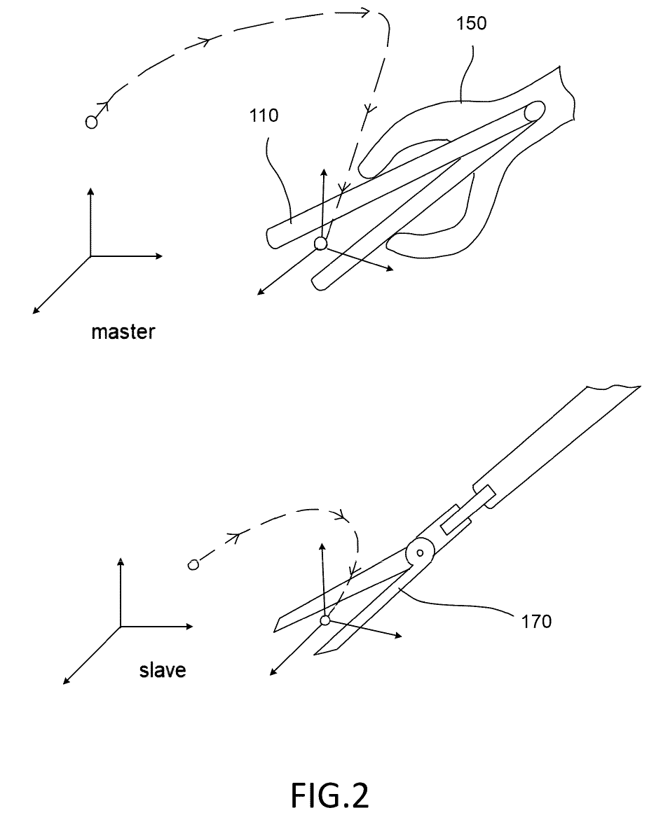

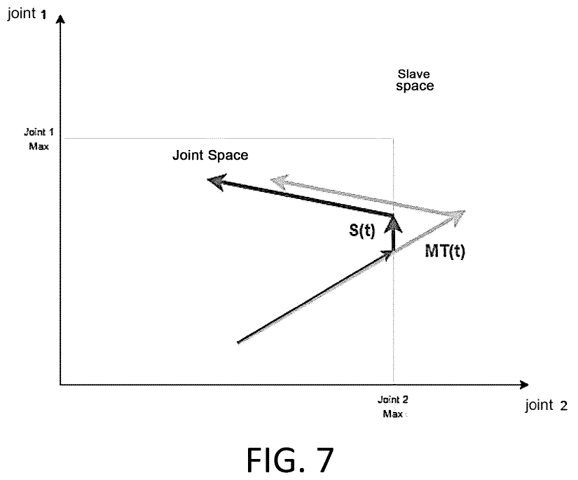

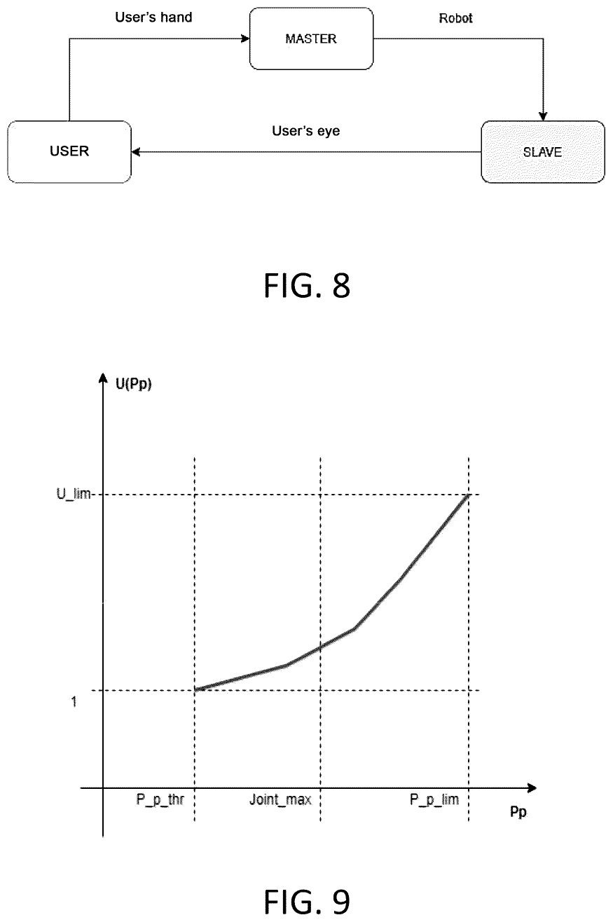

With reference to FIGS. 1-10, a method for controlling a slave device of a robotic system for medical or surgical teleoperation is described.

The robotic system, to which the method applies, comprises at least one master device 110 adapted to be moved by an operator 150, and further comprises at least one slave device comprising a surgical instrument 170 adapted to be controlled by the master device.

The master device 110 is preferably a “wheel” type master device, without force feedback, for mono-lateral teleoperation. Therefore, for example, the master device can be a master mechanically constrained to an operating console and at the same time be of the wheel-type without force feedback, for mono-lateral teleoperation.

The master device 110 is preferably a master device of a type which is mechanically unconstrained to the operating console.

The method comprises the steps of determining, for each master trajectory of the master device, a respective slave target trajectory of the slave device, and controlling the slave device so that it follows and moves along said slave trajectory.

The aforesaid step of determining comprises determining, for each master trajectory of the master device, a respective slave target trajectory of the slave device, in a slave reference coordinate system of the slave device, in which the slave device motions are reduced by a scale factor Fs with respect to the master device motions, and in which the pose of the slave device has a translational offset OFF, which can be zero or non-zero, with respect to the pose of the master device.

The aforesaid slave target trajectory, for any master device motion, is contained in a predetermined convex volume in the slave reference coordinate system.

The aforesaid step of determining the slave target trajectory comprises defining an edge region of said convex volume, close to the boundaries of the convex volume, and an inner region of the convex volume, which is internal with respect to the edge region and thus far from the boundaries of the convex volume.

The method provides, when a slave device nominal target trajectory, corresponding to the master device trajectory mapped in the slave reference coordinate system, is outside the aforesaid inner region of the convex volume, dynamically varying the aforesaid scale factor Fs (also defined hereinafter as “scaling factor”) to obtain a dynamically variable modified scale factor Fm(t), thus modifying the slave trajectory with respect to a nominal slave trajectory which would be obtained with a constant scale factor, so that the slave target trajectory remains within the aforesaid predetermined convex volume.

The method further provides, when the modified scale factor Fm(t) is greater than a predetermined maximum scaling factor Fmax, dynamically modifying also the aforesaid translational offset OFF(t), so that the slave device still remains within the aforesaid predetermined convex volume.

Further details on the scaling factors and translational displacements (or “offsets”) will be shown later in this description.

According to an embodiment of the method, said convex volume corresponds to a workspace of the slave device defined by three orthogonal joints having limited stroke included in the slave device.

In accordance with an embodiment, the method is applied to a slave device comprising at least one joint adapted to move the slave device along at least one respective direction X, Y, Z corresponding to one of the three directions of a spatial reference system X, Y, Z associated with a slave workspace of the slave device.

Such said slave workspace has, in each of the directions X, Y, Z of the aforesaid spatial reference system, a physical limit determined by possible physical movement limits of the slave device in the respective direction.

The aforesaid edge region of the slave workspace comprises the points of the slave workspace which are distant from said physical limits less than a predetermined threshold Xthr, Ythr, Zthr.

In this case, when the aforesaid at least one joint of the slave device is in the edge region, referring to the respective direction, the method provides dynamically varying said scale factor, while the joint approaches the aforesaid physical limits so that the trajectory described by the joint always remains within the slave workspace.

According to an implementation option, the aforesaid physical limits correspond to the coordinates of the maximum limits (Xmax, Ymax, Zmax) and of the minimum limits (Xmin, Ymin, Zmin) of the movement or stroke of the respective joints in the respective direction.

According to an embodiment, the slave device comprises three joints adapted to displace the slave device along a respective one of the three directions of the aforesaid spatial reference system X, Y, Z. The three directions X, Y, Z are orthogonal to one another and define three orthogonal translational degrees of freedom of the joints.

For each of the joints X, Y, Z the physical limits comprise a three-dimensional physical limit determined by the maximum possible physical movement of the device in each of the aforesaid three orthogonal directions.

The aforesaid edge region comprises a space between a first parallelepiped and a second parallelepiped.

The first parallelepiped, corresponding to the aforesaid inner region, is defined by distances corresponding to the predetermined thresholds Xthr, Ythr, Zthr corresponding to the respective directions.

The second parallelepiped is defined by the aforesaid physical movement limits Xmax, Ymax, Zmax corresponding to the respective directions.

The scale factor comprises, for each direction, a respective dynamic scaling function Fsx, Fsy, Fsz.

According to an implementation option, the three dynamic scaling functions Fsx, Fsy, Fsz on the respective directions are mutually equal.

According to another implementation option, the three dynamic scaling functions Fsx, Fsy, Fsz on the respective directions are mutually different.

According to an Implementation option, the aforesaid predetermined thresholds Xthr, Ythr, Zthr are mutually equal, for each limit and/or joint.

According to another implementation option, the aforesaid predetermined thresholds Xthr, Ythr, Zthr, for the different limits and/or joints, are mutually different.

In accordance with an embodiment, the method further comprises the following steps:
defining a nominal target pose in the slave workspace, controlled by a respective master device pose in a workspace of the master device, in the absence of dynamic variations of the scale factor Fs and in the absence of translational offset (OFF);
verifying whether the nominal target pose is inside or outside said inner region of the slave workspace;
if the nominal target pose is outside the aforesaid inner region, the step of dynamically varying the scale factor Fs comprises scaling the scale factor Fs by a reduction function U dependent on the position of the nominal target pose.

According to an implementation option, the aforesaid reduction function U is monotonous and non-decreasing as a function of the distance between the nominal target pose and the nearest point of the inner region.

According to an implementation option, the aforesaid reduction function U acts on each individual joint independently and/or each individual joint operates according to a respective different reduction function U.

In accordance with an implementation option, the reduction function U is an increasing linear function, which has a value 1, corresponding to a nominal scale factor Fs valid for a nominal target pose inside the inner region, at each point belonging to the boundary between the inner region and the edge region, and instead has a value greater than 1 for the points of the edge region, a value which grows when moving away from the boundary between the inner region and the edge region, so that the modified scale factor Fm applied at each point of the edge region grows linearly as a function of the distance of the point from the boundary between the edge region and the inner region.

According to another implementation option, the reduction function U is a non-linear function having the trend of an equilateral hyperbola.

In accordance with an embodiment, when the value of the reduction function U reaches or exceeds the aforesaid predetermined maximum scale factor Fmax, i.e., for the points where the value of the reduction function U reaches or exceeds such a predetermined maximum scaling factor (Fmax), the method provides keeping the slave device target pose stationary, and thus translationally stopping the slave device, so that the target pose deviates from the nominal target pose, and a translational offset ΔOFF is determined between the master device pose and the slave target device pose.

According to an implementation option, the aforesaid predetermined maximum scaling factor Fmax is defined in relation to the definitions of the inner region and the edge region of the slave workspace, so that the controlled trajectory of the slave device exists and extends entirely within the slave workspace of the slave device.

In accordance with an implementation option, when the nominal target pose controlled by the master device is again associated with a reduction function value U lower than the maximum scaling factor Fmax, the method provides remodulating the parameters of the reduction function U so as to recover the accumulated translational offset OFF, when the slave device pose enters again the inner region of the slave workspace.

According to an embodiment, applicable to a slave device which, in addition to translational degrees of freedom corresponding to the aforesaid directions X, Y, Z, provides at least one rotational degree of freedom R representing a rotation of the slave device, at a control point, about the axis thereof, the method comprises controlling the motion of the slave device within the slave device workspace depending on the motion of the master device in the master device workspace, so that the slave device follows the rotation of the master device in the aforesaid at least one rotational degree of freedom with a rotation scale factor Fr.

When such a joint of the slave device is located in the aforesaid inner region, the rotation scale factor Fr is 1.

When such a joint of the slave device is located in the aforesaid edge region, the method provides applying a rotation scale factor greater than 1 and increasing, while the joint approaches the physical limit of the edge region, in a similar manner to what was already shown above with reference to the translational degrees of freedom.

According to another embodiment, the method is applied to a slave device which, in addition to translational degrees of freedom corresponding to the aforesaid directions X, Y, Z, comprises at least two further angular orientation degrees of freedom P, Y which, overall, define the relative orientation of a control point of at least one respective joint of the slave device with respect to the orientation of the master device.

In this case, the method provides that the aforesaid further angular orientation degrees of freedom P, Y are controlled with an orientation scale factor 1:1, so that the orientation of the slave device remains constant and corresponding to the orientation of the master device in whatever position the slave device is located within the slave workspace, even in the edge region.

In accordance with an embodiment of the method, the trajectory of the joint, in the slave workspace, tends to follow the respective instantaneous direction of the trajectory of the master device, in order to maintain the position of the joint always in the allowed slave workspace.

According to an embodiment, the method further comprises the following step: when the slave device is located in the aforesaid inner region of the slave workspace, controlling the movement of the slave device so that it reproduces in every translational direction, in the slave workspace, the movement of the master device in the master workspace, with a constant scale factor not dependent on the position of the slave device.

According to an embodiment of the method, when the master pose controls a slave pose along a direction, the reduction factor U of which is greater than a limit value U_lim (i.e., the scaling factor reaches the maximum value Fmax) the slave device remains motionless along such a direction, thus accumulating a translational offset along such a direction.

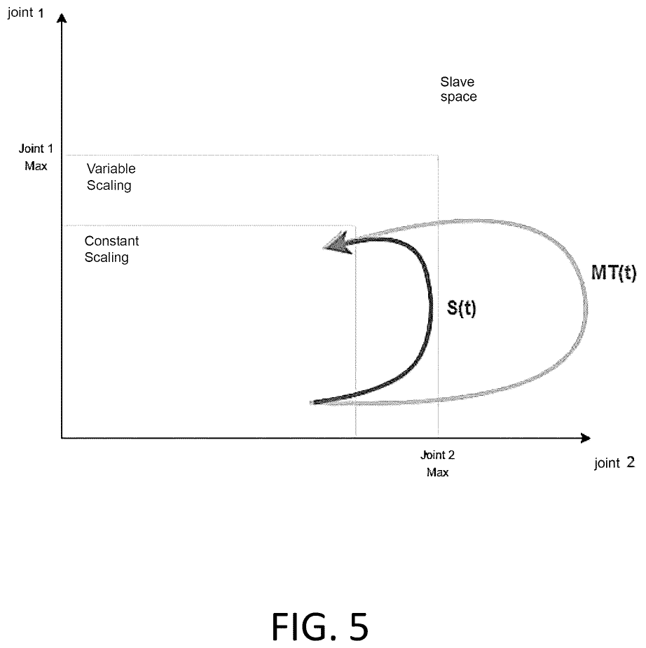

According to an implementation option of the method, shown in FIG. 5, a subspace is defined (corresponding to the aforementioned “edge region”) contained in the physical space of the joints. Such a subspace, defined close to the limits of the workspace, defines an area in which the scale ratio between master and slave is not constant but increases until it becomes infinite on the limits of the joint itself. This solution ensures less distortion of the directions of the target trajectory S with respect to the nominal target trajectory MT (corresponding to the trajectory of the master device mapped in the slave device workspace). Thereby the user perceives more control of the slave device.

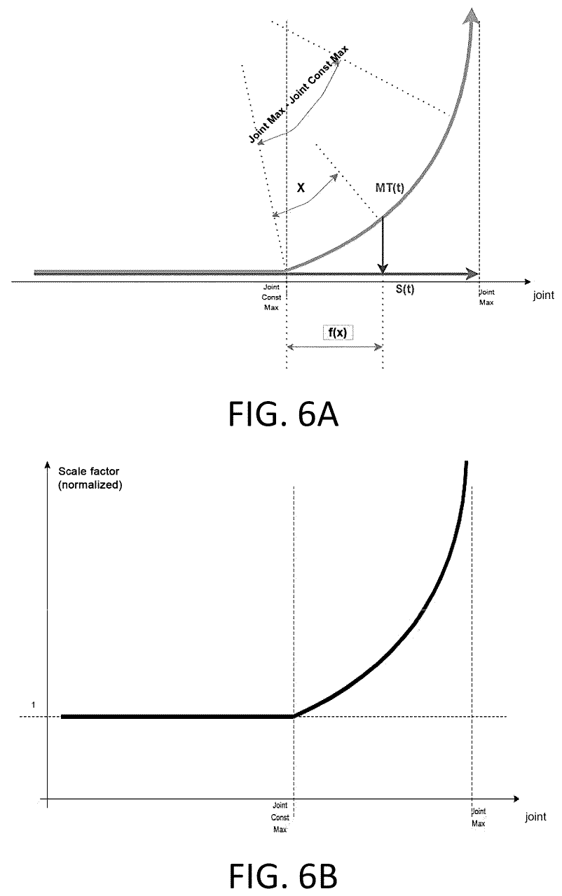

The relationship between the target master MT pose and the slave device S pose in the edge region (with variable scaling) at the level of the single joint can be interpreted as the mapping between a straight line and an equilateral hyperbola, as shown in FIG. 6A.

Similarly, the deformation of the trajectory S(t) with respect to the trajectory MT(t) can be interpreted as the introduction of a dynamic scaling which depends on the current position S(t), as shown in FIG. 6B.

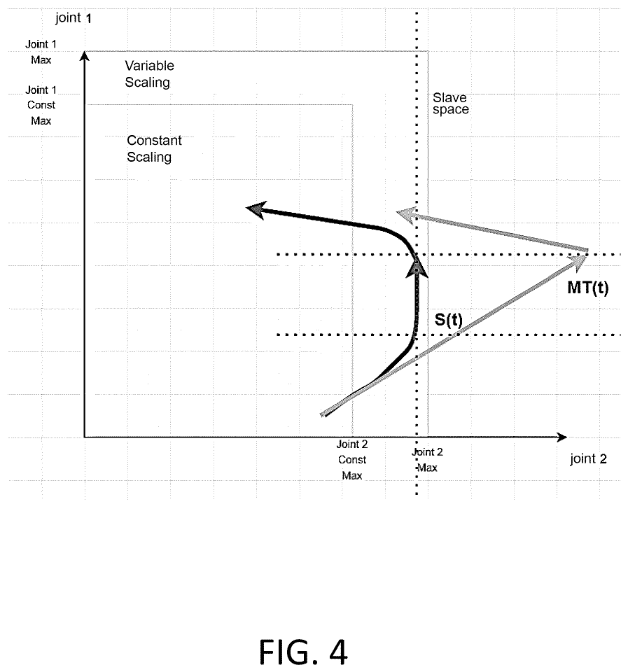

According to another implementation option of the method, shown in FIG. 4, in addition to carrying out the aforesaid dynamic scaling, the method provides that the slave device S(t) starts to again follow trajectories according to the nominal target trajectory (target master trajectory MT(t)) as soon as the master device moves instantaneously in a direction reachable by the slave device. Let's assume that S(t0) is the last valid point of the slave device, MT(t1) the previous target position, and MT(t2) the new target position. At that point S(t2)=S(t0)+(MT(t2)−MT(t1)). If the entire trajectory S(t0) . . . . S(t2) is valid then the Slave S(t) can continue. This solution allows the immediate return to normal situations, at the cost of the introduction of a translational offset which can be used by means of limited teleoperation.

Still referring to FIGS. 1-10, and in particular FIGS. 1-3, a robotic system 100 for medical or surgical teleoperation, included in the present invention, is described below.

Such a robotic system comprises at least one master device 110, adapted to be moved by an operator 150; at least one slave device comprising a surgical instrument 170 adapted to be controlled by the master device; a control unit configured to control the slave device, during a teleoperation, based on the movements of the master device.

The control unit is further configured to carry out the following actions:
determining, for each master trajectory of the master device, a respective slave target trajectory of the slave device;
controlling the slave device so that it follows and moves along the aforesaid slave trajectory.

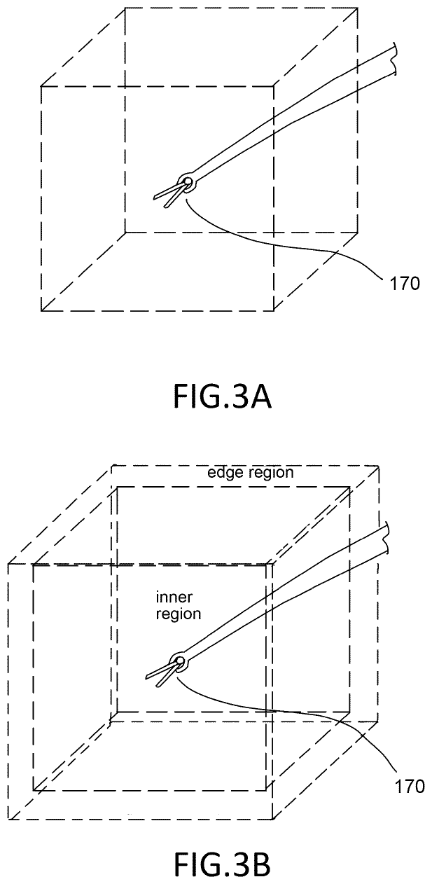

The aforesaid step of determining comprises determining, for each master trajectory of the master device, a respective slave target trajectory of the slave device, in a slave reference coordinate system of the slave device, in which the slave device motions are reduced by a scale factor Fs with respect to the master device motions, and in which the pose of the slave device has a translational offset OFF, which can be zero or non-zero, with respect to the pose of the master device; the aforesaid slave target trajectory, for any master device motion, is contained in a predefined convex volume (shown for example in FIGS. 3A and 3B) in the slave reference coordinate system.

The aforesaid step of determining the slave trajectory further comprises:
defining an edge region of the aforesaid convex volume, close to the boundaries of the convex volume, and an inner region of the convex volume, which is internal with respect to the edge region and thus far from the boundaries of the convex volume;
when a slave device nominal target trajectory, corresponding to the master device trajectory mapped in the slave reference coordinate system, is outside said inner region of the convex volume, dynamically varying the aforesaid scale factor Fs to obtain a dynamically variable modified scale factor Fm(t), thus modifying the slave trajectory with respect to a slave nominal trajectory which would be obtained with a constant scale factor, so that the slave target trajectory remains within the aforesaid predetermined convex volume;
when the modified scale factor Fm(t) is greater than a predetermined maximum scaling factor Fmax, dynamically modifying also such a translational offset OFF(t), so that the slave device still remains within the aforesaid predetermined convex volume.

According to several possible implementation options of the robotic system, the control unit is configured to carry out a method for controlling a slave device according to any one of the embodiments shown in this description.

A mathematical dissertation of an algorithm used in an embodiment of the method is provided below merely by way of a non-limiting exemplary disclosure.

With reference to the aforementioned “poses” of the master and slave devices, it should be noted that, for the purposes of the present disclosure, each “pose” is to be understood as characterized by respective values of the degrees of freedom of the slave device.

Typically, such degrees of freedom comprise 7 degrees of freedom, including three translation degrees of freedom (X, Y, Z), three rotation degrees of freedom, for example the degrees of freedom of roll, pitch and yaw, and an opening/closing degree of freedom (grip).

Thus, a “pose” is defined by respective values of the aforesaid degrees of freedom, and a velocity associated with a pose refers to a velocity of the temporal evolution of a respective degree of freedom; a translational trajectory refers to a translational trajectory in the coordinate system of the translational degrees of freedom X, Y, Z.

Still with reference to poses, the following definitions of “master pose”, “slave pose”, “nominal target pose”, “modified target pose” are used in the present description.

The “master pose” is the current pose of the master device in the reference coordinate system of a master device workspace (e.g., space defined by a tracking mechanism included in the robotic system).

The “slave pose” is the current pose of the slave device in the reference coordinate system of a slave device workspace.

The “nominal target pose” (also sometimes defined in the following as “proxy pose”) is the master device pose mapped in the slave device workspace; it is so referred to because it is the pose which should be tracked by the slave device under “nominal” conditions, i.e., in the absence of further control mechanisms or processing.

It should be noted that the determination of the “nominal target pose” depends solely on translation offsets between the centers of the master and slave reference coordinate systems and the application of the scale factor on the translations. Translation offsets can be defined for example in alignment steps, or by an explicit intervention of the operator, or following the action of usability algorithms.

The “modified target pose” (also sometimes defined in the following as the “target pose”) is the reference pose of the slave device, i.e., the pose to which the slave device must converge following the actuation governed by the control system. This pose can in principle coincide with the nominal target pose, but can also differ therefrom if there are reasons to modify it, by means of specific additional control actions and related algorithms.

In the present description, the modification of the nominal target pose (proxy pose), to obtain the modified target pose (target pose) is performed for example based on information on the current position of the slave device, so as to reduce the delays perceived by the operator between the movement of the slave device and the movement imparted to the master device.

Such a modification can be obtained for example (as will be further illustrated below) by inserting an additional translation offset between proxy pose and target pose.

It is given the master device Pm pose expressed with respect to the reference system of the master device OM, and it is given the target pose of the slave device Ps expressed with respect to the reference system of the slave device OS is given.

In this part of the description, the terminology “slave device pose” is used referring to the “target pose of the slave device Ps”, i.e., the pose upstream of the joint control system, and not to the actual position at time t of the slave device, which could depend on other factors such as the actuation dynamics.

It is assumed, without loss of generality, that OM and OS are both Cartesian reference systems and that the Master Slave teleoperation does not require any transformation between the two reference systems, or (extending then such a consideration to all the axes), that a movement of the master device along the X axis in the reference system OM is translated into a controlled movement of the slave device along the X axis in the reference system OS.

Under these conditions, a translational mapping function is thus defined from the master pose Pm expressed in OM toward the slave target pose Ps expressed in OS. Such a function can be expressed in differential form (in discrete time) by the following equation (EQ1):

$$Ps ( t ) = P s ( t - 1 ) + ( P m ( t ) - Pm ( t - 1 ) ) / Fs$$

$$Ps ( 0 ) = P m ( 0 ) + O F F ( 0 )$$

where Ps(t) and Pm(t) are two column vectors containing the coordinates x,y,z in the respective reference systems at time t; Fs is a diagonal matrix the elements of which are the scale factors along the directions x, y and z; off(0) is a column vector containing the translational offset factors between the reference systems defined at the entry into teleoperation itself so as to minimize the movement of the slave device during the alignment step.

During the course of the teleoperation, it is possible to modify the differential relationship between the motion of the master device and the motion of the slave device expressed in the aforementioned equation (EQ1) by inserting further differential offset factors as in the following equation (EQ2):

$$Ps ( t ) = P s ( t - 1 ) + ( P m ( t ) - P m ( t - 1 ) ) / Fs + O F F ( t )$$

where off(t) is the instantaneous deviation factor (i.e., instantaneous offset) between master pose and slave pose introduced instantaneously at time t due to direct operator intervention or as a result of other algorithms adapted to improve the teleoperation experience.

At time t, it is possible to define a total offset TOT_OFF as the sum of all the instantaneous offsets off(t) from time 0 to time t.

In the embodiment here presented, the method comprises dynamically modifying (i.e., at each instant t) the scale factor Fs and the offset TOT_OFF so that each point of the translational trajectory PS of the slave device, controlled by the master device, always exists within the workspace of the joints of the slave device itself.

In an implementation, the method comprises imposing the permanence of the trajectory PS of the slave device within any predefined convex volume in R3.

The slave workspace (or volume) comprises:
the aforementioned inner region, which, if the slave workspace is defined by orthogonal joints of finite stroke, comprises the points of the slave workspace distant from the aforesaid physical limit by at least a predetermined threshold Xthr, Ythr, Zthr;
the aforementioned edge region, i.e., the region of the workspace which does not belong to the inner region;
a region outside the workspace.

When at least one joint of the slave device is located in the aforesaid edge region, referring to the respective direction, the method provides progressively increasing the aforesaid scale factor, while the joint approaches the relative physical limit, so that the trajectory described by the joint always remains within the slave workspace.

In this context, neglecting without loss of generality the action of other algorithms capable of inserting instantaneous offsets OFF(t), the equation (EQ2) is modified as follows (EQ3):

$$Pp ( t ) = Ps ( t - 1 ) + ( P m ( t ) - P m ( t - 1 ) ) / Fs$$

$$Ps ( t ) = Ps ( t - 1 ) + ( P m ( t ) - P m ( t - 1 ) ) / {Fs}^{′} ( Pp )$$

i.e., a “proxy pose” Pp (or nominal target pose) is calculated in the slave device space considering the scale factor Fs not influenced by the method presented in the present description.

The scaling function Fs′(Pp) is then defined depending on the position of the proxy pose Pp in the reference system OS.

In particular, if the proxy pose Pp is outside the inner region of the workspace, the scale factor is progressively increased so that when the master pose Pm changes there are no slave target poses Ps outside the slave device workspace. It should be noted that the spatial deformation effect occurs both when Pp is located in the edge region (i.e., at points where Pp would be actuatable by the joints of the robot device), and when Pp is outside the workspace of the slave device itself.

In accordance with an embodiment of the method, the dynamic scaling factor Fs′ can thus be defined as follows:

$${Fs}^{′} = Fs * U ( Pp ) \qquad ( EQ4 )$$

where the dependence of the dynamic scale factor on a further reduction factor U dependent on the position of the proxy pose Pp is expressed, In particular, by expressing the dynamic scaling factor Fs' for the joints related to the orthogonal directions XYZ of the robotic system, the following is obtained:

$${Fs}^{′} \_x = Fs\_x * U ( Pp\_x ) \qquad ( EQ5 )$$

$${Fs}^{′} \_y = Fs\_y * U ( Pp\_y ) \qquad ( EQ6 )$$

$${Fs}^{′} \_z = Fs\_z * U ( Pp\_z ) \qquad ( EQ7 )$$

where, according to an embodiment of the method, the formulation of the scalar reduction function u is independent of the axis with respect to which it is applied. The scalar reduction function u is inherently non-decreasing because of the formulation thereof. In other words, the scale factor increases indefinitely as the proxy pose Pp moves away from the inner region of the slave device workspace. The overall effect which is obtained consists of a slowing of the slave device (pose PS) which is observed as it approaches the limit of the workspace.

In this context, a reduction value U_lim is defined beyond which the movement of the slave device following a movement of the master device is no longer perceptible. In other words, a limit scale factor defined (for simplicity in vector form) Fs′_lim:

$${Fs}^{′} \_ lim = Fs * U\_lim \qquad ( EQ8 )$$

The movement obtained by the slave device dPs=Ps(t)−Ps(t−1) following a displacement of the master device dPm=Pm(t)−Pm(t−1) is:

$$dPs = dPm / {Fs}^{′} \_lim$$

and is no longer perceptible by the user in teleoperation and/or causes an unacceptable degradation of teleoperation along such an axis.

In an implementation option of the method, the choice of the value U_lim depends on the scaling factor Fs of the relative axis. Thereby, it is possible to directly control the maximum scale factor described by FS' which the user experiences.

In an implementation option, the method provides, once the scale factor related to an axis reaches the value Fs′_lim, the movements of the master device the target pose of which moves away from the inner region of the workspace do not command any movement of the slave device, i.e., an instantaneous offset off(t) is introduced with respect to the expected movement described by equation EQ3.

In other words, in such an embodiment, equations (EQ2) and (EQ3) are modified as presented by the following algorithm. The following equations refer to the case of the single axis, treated independently as expressed by an embodiment of the method (ALG1).

```
Proxy pose calculation
Pp(t) = Pp(t−1) + (Pm(t) − Pm(t−1))/ Fs
Does the proxy pose map a point in the inner region?
if Pt(t) IN INNER REGION
  Ps(t) = Pp(t) (control to slave device)
otherwise
  Fs′(Pt(t),t) = Fs * U(Pp(t)) (the reduction function is applied);
  if Fs′ < Fs′_lim (scaling factor not too high)
    then off(t) = 0 (no instantaneous offset added),
    otherwise
    OFF(t) = − (Pm(t) − Pm(t−1))/ Fs′ (an offset is inserted to have no
movement)
  Finally, the proxy pose is recalculated taking into account the offset
  Pp(t) = Pp(t−1) + (Pm(t) − Pm(t−1))/Fs + OFF(t)
  Ps(t) = Ps(t−1) + Pm(t) − Pm(1−1)) Fs′ + OFF(t)
```

It should be noted that the dynamic variation of the offset improves the responsiveness of the robotic system at the cost of introducing drifts between the position of the master device and the position of the slave device.

It should also be noted that, due to the insertion of the translational offset upstream of the pose Pp, the movement of such a pose is limited by the quantity Pp_lim defined as the inverse function of U applied in U_lim.

In an implementation of the method, there is a relationship between U_lim (and thus Fs′_lim) and the size of the edge region. In particular, Xthr, Ythr and Zthr must be defined so that the movement of the slave pose Ps is always contained within the slave device workspace. In this context, the quantities Xthr, Ythr and Zthr (which contribute to the definition of Pp_lim) are defined for each joint independently as follows.

U (Pp) is a non-decreasing, active generic function greater than 1 for Pp<Pp_lim and Pp>-Pp_lim as in FIG. 9. Without loss of generality, only the positive semi-axis of Pp is considered.

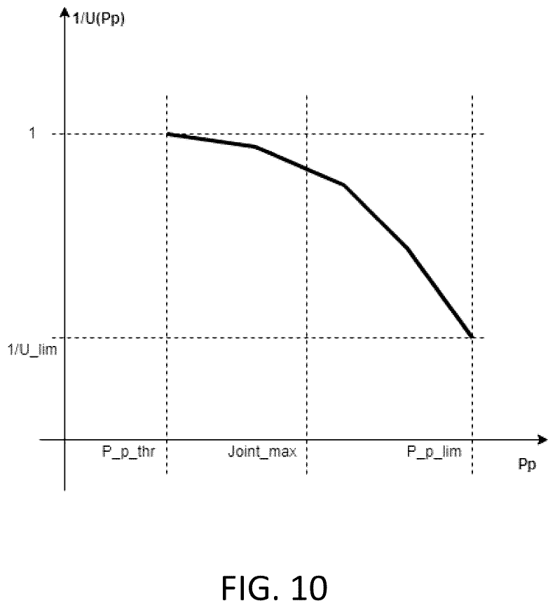

It should be noted that, as shown in FIG. 10, the area under the curve of 1/U (Pp) where Pp is active (between Pp_thr and Pp_lim) is equivalent to the space traveled by the slave device Ps as Pp changes. The existence of Ps within the workspace is thus equivalent to requiring that (EQ 9):

$$Joint\_Max - Pp\_thr > {\int }_{Pp \_ thr}^{Pp \_ lim} U ( Pp ) \partial Pp$$

where Joint_Max is a parameter representative of the maximum possible movement by the respective joint.

In an implementation option, following the saturation of the slave pose as the scale factor is greater than the limit value, when the master device control the proxy pose with a trajectory such as to approach the inner area of the workspace, the shape of the reduction function U is remodulated so as to allow the slave device to recover the accumulated offset once it has returned to the inner region.

In particular, a function U′ is dynamically defined with the following features:
monotone not decreasing
has a value equal to “1” in the outer limit of the inner region of the workspace;
has a value “lim” in the current point of the proxy pose at the moment of return (Pp_lim);
respects the following relationship:

$$Pplim - Ppthr + \sum OFF ( t ) = {\int }_{Ppthr}^{Pplim} {U}^{′} ( Pp ) dPp$$

where Σ(OFF(t)) is the sum of all the instantaneous offsets accumulated since the system entered the saturation step of the slave pose.

According to an embodiment, concerning the case already considered above in which the slave device comprising three joints X, Y, Z, for each of the joints the maximum and minimum physical limits comprise a three-dimensional physical limit determined by the maximum possible physical movement of the device in each of the three orthogonal directions.

Accordingly, the aforesaid edge region comprises a space between a first parallelepiped, referred to as an inner region, defined by distances corresponding to the aforesaid predetermined thresholds Xthr, Ythr, Zthr corresponding to the respective directions, and a second parallelepiped defined by the aforesaid physical movement limits Xmax, Xmin, Ymax, Ymin, Zmax, Zmin corresponding to the respective directions.

The scale factor comprises, for each direction, a respective dynamic scaling function Fs_x′(Pp_x), Fs y′(Pp_y), Fs_z′(Pp_z), which depends on the proxy pose Pp, i.e., the pose which would control the master device in the slave space if the present method were not adopted.

According to an implementation option, the dynamic scaling function related to each of the axes has a minimum limit (maximum scale factor) equal to Fs_lim′. In an implementation option, the scale limit factor is the same for all the axes. According to another implementation option, it depends on the scale factor before applying the method.

In accordance with an embodiment of the method, the aforesaid each dynamic scaling function Fs' assumes the form Fs=Fs*U (Pp), where Fs is the scale factor before the application of the method, and U (Pp) is a reduction factor expressed as a non-increasing function of the distance between the aforesaid proxy pose and the nearest point belonging to the inner region of the slave device workspace defined above.

By virtue of the provision of such a control method, it is possible to avoid exiting the master-slave teleoperation when the pose controlled by the master device is outside the limits of the slave workspace, improving usability.

As can be seen, the objects of the present invention as previously indicated are fully achieved by the method described above by virtue of the features disclosed above in detail.

In order to meet contingent needs, those skilled in the art may make changes and adaptations to the embodiments of the method described above or can replace elements with others which are functionally equivalent, without departing from the scope of the following claims. All the features described above as belonging to a possible embodiment may be implemented irrespective of the other embodiments described.

1. A method for controlling a slave device of a robotic system for medical or surgical teleoperation, wherein said robotic system comprises a master device adapted to be moved by an operator, and a slave device, comprising a surgical instrument adapted to be controlled by the master device, wherein the method comprises:
determining, for each master trajectory of the master device, a respective slave target trajectory of the slave device, in a slave reference coordinate system of the slave device, in which motions of the slave device are reduced by a scale factor with respect to motions of the master device, and in which a pose of the slave device has a translational offset, with respect to a pose of the master device,
wherein said slave target trajectory, for any master device motion, is contained in a predetermined convex volume in the slave reference coordinate system;
controlling the slave device so that the slave device follows and moves along said slave trajectory;
wherein said step of determining the slave target trajectory comprises:
defining an edge region of said convex volume, close to boundaries of the convex volume, and an inner region of the convex volume, which is internal with respect to the edge region and thus apart from the boundaries of the convex volume;
when a slave device nominal target trajectory, corresponding to the master device trajectory mapped in the slave reference coordinate system, is outside said inner region of the convex volume, dynamically varying said scale factor to obtain a dynamically variable modified scale factor, thus modifying the slave trajectory with respect to a nominal slave trajectory which would be obtained with a constant scale factor, so that the slave target trajectory remains within said predetermined convex volume;
when the modified scale factor is greater than a predetermined maximum scale factor, dynamically modifying also said translational offset, so that the slave device remains within said predetermined convex volume.

2. A method according to claim 1, wherein said convex volume corresponds to a workspace of the slave device defined by three orthogonal joints having limited stroke.

3. A method according to claim 1, wherein said at least one slave device comprises at least one joint adapted to displace the slave device along at least one respective direction corresponding to one of three directions of a spatial reference system associated with a slave workspace of the slave device, wherein said slave workspace has physical limits in each of the directions of said spatial reference system determined by limits of possible physical motion of the slave device in the respective direction,
wherein said edge region of the slave workspace comprises points of the slave workspace which are distant from said physical limits less than a predetermined threshold,
wherein, when said at least one joint of the slave device is in said edge region, referring to the respective direction, the method comprises dynamically varying said scale factor, while the joint approaches said physical limits, so that the trajectory described by the joint always remains within the slave workspace.

4. A method according to claim 3, wherein the slave device comprises three joints adapted to displace the slave device along a respective one of the three directions of said spatial reference system, wherein said three directions are orthogonal to one another and define three orthogonal translational degrees of freedom of the joints,
wherein, for each of the joints associated with said directions the physical limits comprise a three-dimensional physical limit determined by a maximum possible physical motion of the device in each of said three orthogonal directions,
wherein said edge region comprises a space between a first parallelepiped corresponding to said inner region, defined by distances corresponding to said predetermined thresholds corresponding to the respective directions, and a second parallelepiped defined by said physical movement limits corresponding to the respective directions,
wherein said scale factor comprises, for each direction, a respective dynamic scaling function, wherein the three dynamic scaling functions on the respective directions are the same or are different from each other,
wherein the predetermined thresholds are the same or are different from each other for each limit and/or joint.

5. A method according to claim 1, further comprising:
defining a nominal target pose in the slave workspace, controlled by a respective master device pose in a workspace of the master device, in absence of said dynamic variations of the scale factor and in absence of said translational offset;
verifying whether the nominal target pose is inside or outside said inner region of the convex volume of the slave workspace;
if the nominal target pose is outside said inner region, said step of dynamically varying the scale factor comprises scaling the scale factor by a reduction function dependent on the position of the nominal target pose.

6. A method according to claim 5, wherein said reduction function is monotonous and non-decreasing as a function of the distance between the nominal target pose and a nearest point of the inner region.

7. A method according to claim 5, wherein said reduction function acts on each individual joint independently and/or wherein each individual joint operates according to a respective different reduction function.

8. A method according to claim 6, wherein the reduction function is an increasing linear function, which has a value 1, corresponding to a nominal scale factor valid for a nominal target pose inside the inner region, at each point belonging to the boundary between the inner region and the edge region, and a value greater than 1 and growing for the points of the edge region when moving away from the boundary between the inner region and the edge region, so that the modified scale factor applied at each point of the edge region grows linearly as a function of a distance of the point from the boundary between the edge region and the inner region.

9. A method according to claim 6, wherein the reduction function is a non-linear function having the trend of an equilateral hyperbola.

10. A method according to claim 5, wherein when the value of the reduction function reaches or exceeds said predetermined maximum scale factor for points where the value of the reduction function reaches or exceeds said predetermined maximum scaling factor, the method comprises keeping the slave device target pose stationary, and thus translationally stopping the slave device, so that the target pose deviates from the nominal target pose, and a translational offset is determined between the master device pose and the slave target device pose.

11. A method according to claim 10, wherein said predetermined maximum scaling factor is defined in relation to definitions of the inner region and the edge region of the slave workspace, so that a controlled trajectory of the slave device exists and extends entirely within the slave workspace of the slave device.

12. A method according to claim 6, wherein when the nominal target pose controlled by the master device is associated with a reduction function value lower than said maximum scaling factor, the method comprises remodulating the parameters of the reduction function to recover the accumulated translational offset when the slave device pose enters again the inner region of the slave workspace.

13. A method according to claim 1, wherein the slave device, in addition to translational degrees of freedom corresponding to said directions, comprises at least one rotational degree of freedom representing a rotation of the slave device, at a control point, about an axis thereof,
wherein the method comprises controlling the slave device motion within the slave device workspace as a function of the master device motion in the master device workspace, so that the slave device follows the rotation of the master device in said at least one rotation degree of freedom with a rotation scale factor,
wherein, when said slave device joint is in said inner region, the rotation scale factor is equal to 1, and wherein, when said slave device joint is in said edge region, the method comprises applying an increasing rotation scale factor greater than 1, while the joint approaches the physical limit of the edge region.

14. A method according to claim 1, wherein said master device is a groundless-type master device;
and/or wherein said master device is a master device of the type which is mechanically unconstrained to an operating console.

15. A robotic system for medical or surgical teleoperation, comprising:
at least one master device adapted to be moved by an operator;
at least one slave device comprising a surgical instrument adapted to be controlled by the master device;
a control unit configured to control the slave device, during a teleoperation, based on master device movements,
wherein the control unit is further configured for:
determining, for each master trajectory of the master device, a respective slave target trajectory of the slave device, in a slave reference coordinate system of the slave device, in which the slave device motions are reduced by a scale factor with respect to the master device motions, and in which a pose of the slave device has a translational offset, with respect to a pose of the master device, wherein said slave target trajectory, for any master device motion, is contained in a predetermined convex volume in the slave reference coordinate system;
controlling the slave device so that the slave devices follows and moves along said slave trajectory;
wherein said step of determining the slave target trajectory comprises:
defining an edge region of said convex volume, close to boundaries of the convex volume, and an inner region of the convex volume, which is internal with respect to the edge region and thus apart from the boundaries of the convex volume;
when a slave device nominal target trajectory, corresponding to the master device trajectory mapped in the slave reference coordinate system, is outside said inner region of the convex volume, dynamically varying said scale factor to obtain a dynamically variable modified scale factor, thus modifying the slave trajectory with respect to a slave nominal trajectory which would be obtained with a constant scale factor, so that the slave target trajectory remains within said predetermined convex volume;
when the modified scale factor is greater than a predetermined maximum scale factor, dynamically modifying also said translational offset, so that the slave device remains within said predetermined convex volume.

16. (canceled)

17. A method according to claim 1, wherein said master device is a groundless-type master device, without force return;
and/or wherein said master device is a master device of the type which is mechanically unconstrained to an operating console.
